(19) United States
(12) Patent Application Publication
(10) Pub. No.: US 2024/0169118 A1
(43) Pub. Date: May 23, 2024

(54) METHOD AND DEVICE FOR DESIGNING INSULATOR STRUCTURE FOR MIXED GAS INSULATION

(71) Applicants: GUANGDONG POWER GRID CO., LTD., Guangzhou (CN); ELECTRIC POWER RESEARCH INSTITUTE OF GUANGDONG POWER GRID CO., LTD., Guangzhou (CN)

(72) Inventors: Nian Tang, Guangzhou (CN); Li Li, Guangzhou (CN); Wenguo Gu, Guangzhou (CN); Dongwei Sun, Guangzhou (CN); Zhi Li, Guangzhou (CN); Manjun Zhang, Guangzhou (CN)

(21) Appl. No.: 18/399,679

(22) Filed: Dec. 28, 2023

Related U.S. Application Data
(63) Continuation-in-part of application No. PCT/CN23/72700, filed on Jan. 17, 2023.

(30) Foreign Application Priority Data
Nov. 17, 2022 (CN) 202211442811.8

(51) Int. Cl. G06F 30/20

(52) U.S. Cl. CPC G06F 30/20

(57) ABSTRACT

Disclosed are a method and device for designing an insulator structure for fixed gas insulation. The present disclosure inputs a plurality of sets of pre-set insulator structure parameters into an initial insulator model separately to generate a plurality of insulator models; evaluates an electrical parameter of each insulator model at a same temperature to obtain an electric field distribution of each part of each insulator model; selects a target insulator model from all the insulator models based on a pre-defined model screening condition and the electric field distribution of each part of each insulator model; evaluates a thermodynamic parameter of the target insulator model based on conductivity-temperature variation curves of an insulation material and an insulation gas to obtain a temperature distribution of each part of the target insulator model; and optimizes the target insulator model based on the temperature distribution to obtain an optimal insulator model.

### CROSS REFERENCE TO RELATED APPLICATIONS

The present application is a Continuation-In-Part Application of PCT Application No. PCT/CN2023/072700 filed on Jan. 17, 2023, which claims the benefit of Chinese Patent Application No. 202211442811.8 filed on Nov. 17, 2022. All the above are hereby incorporated by reference in their entirety.

### TECHNICAL FIELD

The present disclosure relates to the technical field of power systems, and in particular, to a method and device for designing an insulator structure for mixed gas insulation.

### BACKGROUND

In the power industry, sulfur hexafluoride (SF₆) is widely used in gas insulated electrical devices due to its excellent insulation and arc extinguishing performance. Considering that SF₆ is a serious greenhouse gas, an environmentally-friendly insulation medium is gradually being adopted to replace SF₆ to limit use of SF₆. However, how to design a structure of an insulator to ensure that the insulator can meet a practical engineering requirement under an operating condition of using an environmentally-friendly mixed gas as an insulation medium has become a major problem that urgently needs to be resolved.

### SUMMARY

In order to overcome the defects in the prior art, the present disclosure provides a method and device for designing an insulator structure for mixed gas insulation, to ensure that an insulator can meet a practical engineering requirement under an operating condition of using an environmentally-friendly mixed gas as an insulation medium.

To resolve the foregoing technical problems, according to a first aspect, an embodiment of the present disclosure provides a method for designing an insulator structure for mixed gas insulation. The method includes:
obtaining a plurality of sets of pre-set insulator structure parameters, and inputting each set of insulator structure parameters into an initial insulator model separately to generate a plurality of insulator models;
evaluating an electrical parameter of each of the insulator models at a same temperature to obtain an electric field distribution of each part of each of the insulator models;
selecting a target insulator model from all the insulator models based on a pre-defined model screening condition and the electric field distribution of each part of each of the insulator models;
evaluating a thermodynamic parameter of the target insulator model based on conductivity-temperature variation curves of an insulation material and an insulation gas to obtain a temperature distribution of each part of the target insulator model; and
optimizing the target insulator model based on the temperature distribution of each part of the target insulator model to obtain an optimal insulator model, and obtaining an insulator based on the optimal insulator model.

Further, the evaluating an electrical parameter of each of the insulator models at a same temperature to obtain an electric field distribution of each part of each of the insulator models specifically includes:
calculating tangential electric field strength, normal electric field strength, and total electric field strength of each of the insulator models separately at the same temperature to obtain the electric field distribution of each part of each of the insulator models.

Further, the calculating tangential electric field strength of the insulator model specifically includes:
when it is detected that a surface charge accumulation of the insulator model reaches a maximum value, calculating a maximum value of a tangent vector of a surface electric field of the insulator model, and using the maximum value of the tangent vector of the surface electric field as the tangential electric field strength.

Further, the target insulator model is an insulator model with minimum electric field strength at a triple junction of metal, the insulation gas and the insulation material, and surface electric field strength less than a preset threshold.

Further, the evaluating a thermodynamic parameter of the target insulator model based on conductivity-temperature variation curves of an insulation material and an insulation gas to obtain a temperature distribution of each part of the target insulator model specifically includes:
calculating a temperature characteristic of the target insulator model based on the electric field distribution of each part of the target insulator model and the conductivity-temperature variation curves of the insulation material and the insulation gas to obtain the temperature distribution of each part of the target insulator model.

According to a second aspect, an embodiment of the present disclosure provides a device for designing an insulator structure for mixed gas insulation. The device includes:
an insulator model generation module configured to obtain a plurality of sets of pre-set insulator structure parameters, and input each set of insulator structure parameters into an initial insulator model separately to generate a plurality of insulator models;
an electrical parameter evaluation module configured to evaluate an electrical parameter of each of the insulator models at a same temperature to obtain an electric field distribution of each part of each of the insulator models;
an insulator model screening module configured to select a target insulator model from all the insulator models based on a pre-defined model screening condition and the electric field distribution of each part of each of the insulator models;
a thermodynamic parameter evaluation module configured to evaluate a thermodynamic parameter of the target insulator model based on conductivity-temperature variation curves of an insulation material and an insulation gas to obtain a temperature distribution of each part of the target insulator model; and
an insulator model optimization module configured to optimize the target insulator model based on the temperature distribution of each part of the target insulator model to obtain an optimal insulator model, and obtain an insulator based on the optimal insulator model.

Further, the electrical parameter evaluation module is specifically configured to: calculate tangential electric field strength, normal electric field strength, and total electric field strength of each of the insulator models separately at the same temperature to obtain the electric field distribution of each part of each of the insulator models.

Further, the electrical parameter evaluation module includes:
a tangential electric field strength calculation unit configured to: when it is detected that a surface charge accumulation of the insulator model reaches a maximum value, calculate a maximum value of a tangent vector of a surface electric field of the insulator model, and use the maximum value of the tangent vector of the surface electric field as the tangential electric field strength.

Further, the target insulator model is an insulator model with minimum electric field strength at a triple junction of metal, the insulation gas and the insulation material, and surface electric field strength less than a preset threshold.

Further, the thermodynamic parameter evaluation module is specifically configured to calculate a temperature characteristic of the target insulator model based on the electric field distribution of each part of the target insulator model and the conductivity-temperature variation curves of the insulation material and the insulation gas to obtain the temperature distribution of each part of the target insulator model.

The embodiments of the present disclosure have following beneficial effects:

A plurality of sets of pre-set insulator structure parameters are separately input into an initial insulator model to generate a plurality of insulator models. An electrical parameter of each of the insulator models is evaluated at a same temperature to obtain an electric field distribution of each part of each of the insulator models. A target insulator model is selected from all the insulator models based on a pre-defined model screening condition and the electric field distribution of each part of each of the insulator models. A thermodynamic parameter of the target insulator model is evaluated based on conductivity-temperature variation curves of an insulation material and an insulation gas to obtain a temperature distribution of each part of the target insulator model. The target insulator model is optimized based on the temperature distribution of each part of the target insulator model to obtain an optimal insulator model, and an insulator is obtained based on the optimal insulator model. This can ensure that the insulator meets a practical engineering requirement under an operating condition of using an environmentally-friendly mixed gas as an insulation medium.

### BRIEF DESCRIPTION OF THE DRAWINGS

FIG. 1 is a schematic flowchart of a method for designing an insulator structure for mixed gas insulation according to a first embodiment of the present disclosure;

FIG. 2 is a schematic structural diagram of an example initial insulator model according to a first embodiment of the present disclosure;

FIG. 3 is a schematic structural diagram of an example optimal insulator model according to a first embodiment of the present disclosure;

FIG. 4 is a schematic diagram of electric field strength of an upper surface of an example optimal insulator model according to a first embodiment of the present disclosure;

FIG. 5 is a schematic diagram of electric field strength of a lower surface of an example optimal insulator model according to a first embodiment of the present disclosure; and

FIG. 6 is a schematic structural diagram of a device for designing an insulator structure for mixed gas insulation according to a second embodiment of the present disclosure.

### DETAILED DESCRIPTION

The technical solutions in the present disclosure are clearly and completely described below with reference to the accompanying drawings in the present disclosure. Apparently, the described embodiments are only a part rather than all of the embodiments of the present disclosure. All other embodiments obtained by those skilled in the art based on the embodiments of the present disclosure without creative efforts shall fall within the protection scope of the present disclosure.

It should be noted that step numbers in this specification are only intended to facilitate explanation of specific embodiments, and are not used to limit the sequence of steps. A method provided in the embodiments may be executed by a related terminal device, and the following description is made by taking a processor as an execution main body.

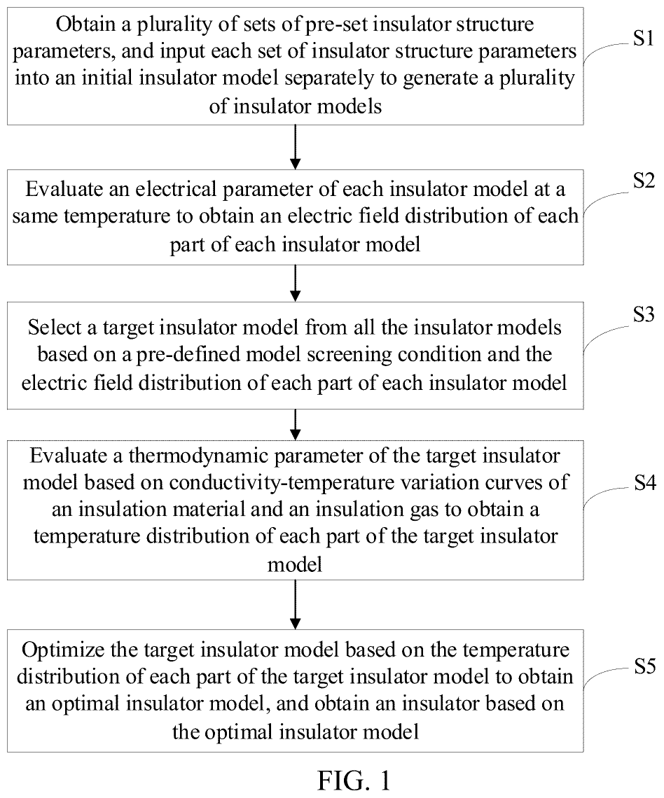

As shown in FIG. 1, a first embodiment of the present disclosure provides a method for designing an insulator structure for mixed gas insulation. The method includes steps S1 to S5:

S1: Obtain a plurality of sets of pre-set insulator structure parameters, and input each set of insulator structure parameters into an initial insulator model separately to generate a plurality of insulator models.

S2: Evaluate an electrical parameter of each insulator model at a same temperature to obtain an electric field distribution of each part of each insulator model.

S3: Select a target insulator model from all the insulator models based on a pre-defined model screening condition and the electric field distribution of each part of each insulator model.

S4: Evaluate a thermodynamic parameter of the target insulator model based on conductivity-temperature variation curves of an insulation material and an insulation gas to obtain a temperature distribution of each part of the target insulator model.

S5: Optimize the target insulator model based on the temperature distribution of each part of the target insulator model to obtain an optimal insulator model, and obtain an insulator based on the optimal insulator model.

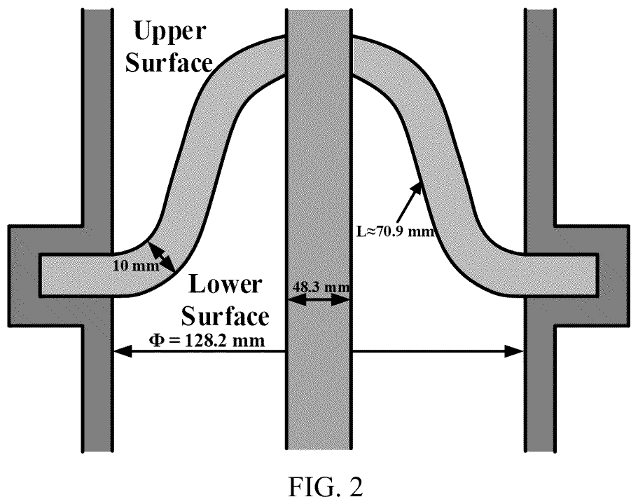

As an example, in the step S1, the initial insulator model is constructed in advance based on a practical engineering requirement. A schematic structural diagram of the initial insulator model is shown in FIG. 2. The plurality of sets of insulator structure parameters are pre-set. The insulator structure parameters include at least one of a radius of angle of rotation of a flange, a clearance between the insulator and the flange, an insulator thickness, an inclination angle of the insulator, a distance between a tail end of the flange and the insulator, and an edge radius of the insulator close to a ground end. Each set of insulator structure parameters is input into the initial insulator model separately to generate the plurality of insulator models.

In the step S2, a temperature is fixedly set based on the practical engineering requirement. At the same temperature, the electrical parameter of each insulator model is evaluated, in other words, the electric field distribution is calculated, to obtain the electric field distribution of each part of each insulator model.

In the step S3, in order to select an insulator model with a structure applicable to an electrical characteristic, namely, the target insulator model, from all the insulator models, the model screening condition is pre-defined. The target insulator model is selected from all the insulator models based on the pre-defined model screening condition and the electric field distribution of each part of each insulator model.

In the step S4, the conductivity-temperature variation curves of the insulation material and the insulation gas are obtained. Based on the conductivity-temperature variation curves of the insulation material and the insulation gas, the thermodynamic parameter of the target insulator model is evaluated, in other words, a temperature characteristic of the target insulator model is calculated, to obtain the temperature distribution of each part of the target insulator model.

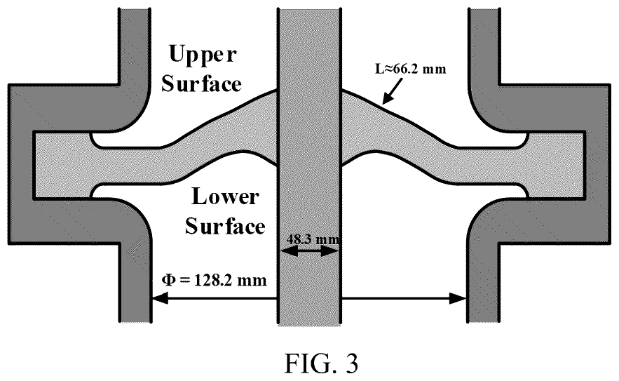

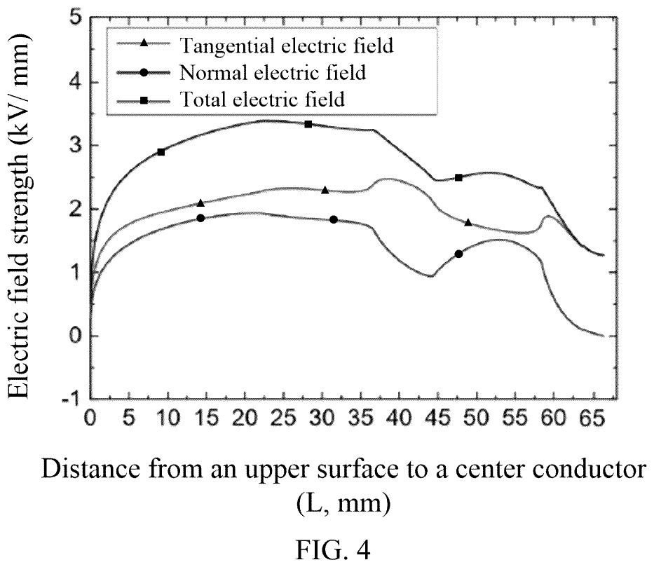

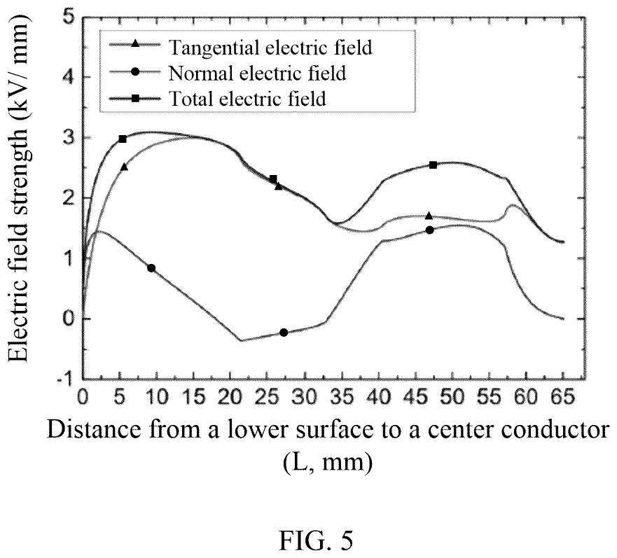

In the step S5, the target insulator model is optimized based on the temperature distribution of each part of the target insulator model, including adjustment of insulator structure parameters of the target insulator model, such that the optimal insulator model is obtained. Then a structural design of the insulator is completed. A schematic structural diagram of the optimal insulator model is shown in FIG. 3, and a schematic diagram of electric field strength of the optimal insulator model is shown in FIG. 4 and FIG. 5.

This embodiment ensures that the insulator made based on the optimal insulator model meets the practical engineering requirement under an operating condition of using an environmentally-friendly mixed gas as an insulation medium.

In a preferred embodiment, the evaluating an electrical parameter of each insulator model at a same temperature to obtain an electric field distribution of each part of each insulator model specifically includes: calculating tangential electric field strength, normal electric field strength, and total electric field strength of each insulator model separately at the same temperature to obtain the electric field distribution of each part of the insulator model.

As an example, for each insulator model, a temperature T1 at a center conductor is fixedly set to 30° C., and a temperature T2 at a grounding point of an outer shell is fixedly set to 30° C. Under the above set temperatures, the tangential electric field strength, the normal electric field strength, and the total electric field strength of the insulator model are calculated separately to obtain the electric field distribution of each part of the insulator model. The electric field strength E is calculated according to following formulas:

div\vec{J_{c}}=div(σ\vec{E})=0 (1)

\vec{E}=−gradV (2)

In the formulas (1) and (2), \vec{J_{c}} represents a current density (A/m²), σ represents conductivity (S/m), V represents an electric potential. A boundary condition is a Dirichlet boundary condition, with V=0 on a low voltage side and V=Utest on a high voltage side, where Utest represents a test voltage applied when the electric field distribution is tested.

In a process of calculating the electric field strength, a phenomenon of surface charge accumulation should also be considered, and a steady-state surface charge accumulation of the insulator model is calculated. The steady-state surface charge accumulation ρ is calculated according to a following formula:

ρₛ=ε₀ε₁E₁−ε₀ε₂E₂ (3)

In the formula (3), ε₀ represents a vacuum dielectric constant, ε₁ represents a relative dielectric constant of the insulation gas, ε₂ represents a relative dielectric constant of the insulation material, and E₁ and E₂ respectively represent normal electric field strength on a gas side and an insulation component side near a surface of the insulator model.

In this embodiment, the tangential electric field strength, the normal electric field strength, and the total electric field strength of the insulator model are calculated separately at the same temperature to obtain the electric field distribution of each part of the insulator model. This can improve accuracy of calculating the electric field distribution.

In a preferred embodiment, the calculating tangential electric field strength of the insulator model specifically includes: when it is detected that a surface charge accumulation of the insulator model reaches a maximum value, calculating a maximum value of a tangent vector of a surface electric field of the insulator model, and using the maximum value of the tangent vector of the surface electric field as the tangential electric field strength.

As an example, when the surface charge accumulation of the insulator model reaches dynamic equilibrium, the surface charge accumulation of the insulator model reaches the maximum value. Therefore, whether the surface charge accumulation of the insulator model reaches the maximum value can be detected by monitoring whether the surface charge accumulation of the insulator model reaches the dynamic equilibrium. When it is detected that the surface charge accumulation of the insulator model reaches the maximum value, the maximum value of the tangent vector of the surface electric field of the insulator model can be calculated and used as the tangential electric field strength.

In this embodiment, when the surface charge accumulation of the insulator model reaches the maximum value, the maximum value of the tangent vector of the surface electric field of the insulator model is calculated and used as the tangential electric field strength. This can further improve the accuracy of calculating the electric field distribution.

In a preferred embodiment, the target insulator model is an insulator model with minimum electric field strength at a triple junction of metal, the insulation gas and the insulation material, and surface electric field strength less than a preset threshold.

As an example, the structure applicable to the electrical characteristic should meet two requirements: 1. The electric field strength at the triple junction of the metal, the insulation gas and the insulation material should be minimum. 2. The surface electric field strength should be kept as small as possible to minimize the surface charge accumulation. In order to select the insulator model with the structure applicable to the electrical characteristic from all the insulator models, the model screening condition is pre-defined as that the electric field strength at the triple junction of the metal, the insulation gas and the insulation material is minimum and the surface electric field strength is less than the preset threshold. Based on the electric field distribution of each part of each insulator model, the insulator model with the minimum electric field strength at the triple junction of the metal, the insulation gas and the insulation material, and the surface electric field strength less than the preset threshold is selected from all the insulator models to obtain the target insulator model.

In this embodiment, the insulator model with the minimum electric field strength at the triple junction of the metal, the insulation gas and the insulation material, and the surface electric field strength less than the preset threshold is used as the target insulator model through screening. This can effectively ensure that a subsequently designed insulator structure is applicable to the electrical characteristic.

In a preferred embodiment, the evaluating a thermodynamic parameter of the target insulator model based on conductivity-temperature variation curves of an insulation material and an insulation gas to obtain a temperature distribution of each part of the target insulator model specifically includes: calculating the temperature characteristic of the target insulator model based on the electric field distribution of each part of the target insulator model and the conductivity-temperature variation curves of the insulation material and the insulation gas to obtain the temperature distribution of each part of the target insulator model.

As an example, the conductivity-temperature variation curves of the insulation material and the insulation gas are obtained according to a following formula:

σ(E,T)=σ₀e^{-W/KT}e^{βE} (4)

In the formula (4), σ(.) represents conductivity of the insulation material used for the insulator; E represents the electric field strength; T represents the temperature; W represents thermal activation energy, which is set to 0.95 eV; K represents a Boltzmann constant, which is set to 8.62×10−5 eV/K; β represents a field dependence coefficient, which is set to 0.08 mm/kV; and σ₀ is a constant, which is set to 19.9 S/m.

The temperature characteristic of the target insulator model is calculated based on the known electric field distribution of each part of the target insulator model and the conductivity-temperature variation curves of the insulation material and the insulation gas to obtain the temperature distribution of each part of the target insulator model. In a process of calculating the temperature characteristic, an equation for solving thermal equilibrium is as follows:

div(k,gradT)=0 (5)

In the formula (5), K represents a thermal conductivity coefficient (W/m·K), T represents the temperature, and the boundary condition is the Dirichlet boundary conditions, with T=70° C. on the low voltage side and T=105° C. on the high voltage side.

In this embodiment, the temperature characteristic of the target insulator model is calculated based on the conductivity-temperature variation curves of the insulation material and the insulation gas to obtain the temperature distribution of each part of the target insulator model. Comprehensive performance of the designed insulator structure can be evaluated based on the electric field distribution and the temperature distribution of the target insulator model for adaptive optimization, thereby ensuring that the insulator meets the practical engineering requirement under the operating condition of using the environmentally-friendly mixed gas as the insulation medium.

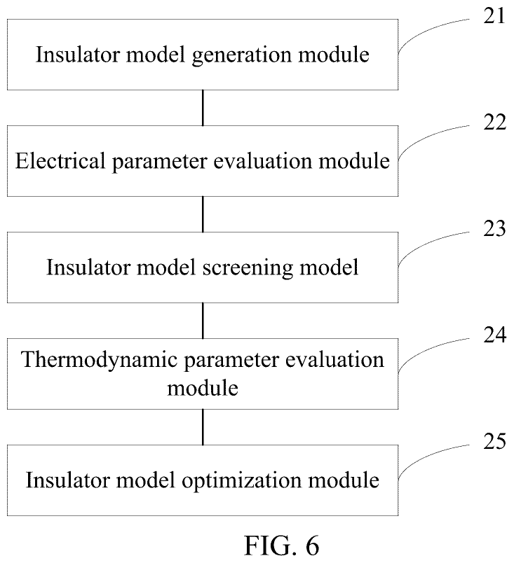

Based on the same inventive concept of the first embodiment, a second embodiment provides a device for designing an insulator structure for mixed gas insulation, as shown in FIG. 6. The device includes: an insulator model generation module 21 configured to obtain a plurality of sets of pre-set insulator structure parameters, and input each set of insulator structure parameters into an initial insulator model to generate a plurality of insulator models; an electrical parameter evaluation module 22 configured to evaluate an electrical parameter of each insulator model at a same temperature to obtain an electric field distribution of each part of each insulator model; an insulator model screening module 23 configured to select a target insulator model from all the insulator models based on a pre-defined model screening condition and the electric field distribution of each part of each insulator model; a thermodynamic parameter evaluation module 24 configured to evaluate a thermodynamic parameter of the target insulator model based on conductivity-temperature variation curves of an insulation material and an insulation gas to obtain a temperature distribution of each part of the target insulator model; and an insulator model optimization module 25 configured to optimize the target insulator model based on the temperature distribution of each part of the target insulator model to obtain an optimal insulator model, and obtain an insulator based on the optimal insulator model.

In a preferred embodiment, the electrical parameter evaluation module 22 is specifically configured to: for each insulator model, calculate tangential electric field strength, normal electric field strength, and total electric field strength of the insulator model separately at the same temperature to obtain the electric field distribution of each part of the insulator model.

In a preferred embodiment, the electrical parameter evaluation module 22 includes: a tangential electric field strength calculation unit configured to: when it is detected that a surface charge accumulation of the insulator model reaches a maximum value, calculate a maximum value of a tangent vector of a surface electric field of the insulator model, and use the maximum value of the tangent vector of the surface electric field as the tangential electric field strength.

In a preferred embodiment, the target insulator model is an insulator model with minimum electric field strength at a triple junction of metal, the insulation gas and the insulation material, and surface electric field strength less than a preset threshold.

In a preferred embodiment, the thermodynamic parameter evaluation module 24 is specifically configured to calculate a temperature characteristic of the target insulator model based on the electric field distribution of each part of the target insulator model and the conductivity-temperature variation curves of the insulation material and the insulation gas to obtain the temperature distribution of each part of the target insulator model.

In another implementation example, the above device for designing an insulator structure for mixed gas insulation includes a processor. The processor is configured to execute the program modules and units stored in a memory, including the insulator model generation module 21, the electrical parameter evaluation module 22, the insulator model screening module 23, the thermodynamic parameter evaluation module 24, the insulator model optimization module 25, and the tangential electric field strength calculation unit.

To sum up, the embodiments of the present disclosure have following beneficial effects:

A plurality of sets of pre-set insulator structure parameters are put into an initial insulator model separately to generate a plurality of insulator models. An electrical parameter of each insulator model is evaluated at a same temperature to obtain an electric field distribution of each part of each insulator model. A target insulator model is selected from all the insulator models based on a pre-defined model screening condition and the electric field distribution of each part of each insulator model. A thermodynamic parameter of the target insulator model is evaluated based on conductivity-temperature variation curves of an insulation material and an insulation gas to obtain a temperature distribution of each part of the target insulator model. The target insulator model is optimized based on the temperature distribution of each part of the target insulator model to obtain an optimal insulator model. This can ensure that an insulator meets a practical engineering requirement under an operating condition of using an environmentally-friendly mixed gas as an insulation medium.

The descriptions above are preferred implementations of the present disclosure. It should be noted that for a person of ordinary skill in the art, various improvements and modifications can be made without departing from the principles of the present disclosure. These improvements and modifications should also be regarded as falling into the protection scope of the present disclosure.

A person of ordinary skill in the art can understand that all or some of processes for implementing the foregoing embodiments can be completed by a computer program instructing relevant hardware. The program may be stored in a computer-readable storage medium. When the program is executed, the processes of the foregoing embodiments may be performed. The storage medium may be a magnetic disk, an optical disc, a read-only memory (ROM), a random access memory (RAM), or the like.

1. A method for designing an insulator structure for mixed gas insulation, comprising:
obtaining a plurality of sets of pre-set insulator structure parameters, and inputting each set of insulator structure parameters into an initial insulator model separately to generate a plurality of insulator models;
evaluating an electrical parameter of each of the insulator models at a same temperature to obtain an electric field distribution of each part of each of the insulator models;
selecting a target insulator model from all the insulator models based on a pre-defined model screening condition and the electric field distribution of each part of each of the insulator models;
evaluating a thermodynamic parameter of the target insulator model based on conductivity-temperature variation curves of an insulation material and an insulation gas to obtain a temperature distribution of each part of the target insulator model; and
optimizing the target insulator model based on the temperature distribution of each part of the target insulator model to obtain an optimal insulator model, and obtaining an insulator based on the optimal insulator model.

2. The method for designing an insulator structure for mixed gas insulation according to claim 1, wherein the evaluating an electrical parameter of each of the insulator models at a same temperature to obtain an electric field distribution of each part of each of the insulator models specifically comprises:
calculating tangential electric field strength, normal electric field strength, and total electric field strength of each of the insulator models separately at the same temperature to obtain the electric field distribution of each part of each of the insulator models.

3. The method for designing an insulator structure for mixed gas insulation according to claim 2, wherein the calculating tangential electric field strength of the insulator model specifically comprises:
when it is detected that a surface charge accumulation of the insulator model reaches a maximum value, calculating a maximum value of a tangent vector of a surface electric field of the insulator model, and using the maximum value of the tangent vector of the surface electric field as the tangential electric field strength.

4. The method for designing an insulator structure for mixed gas insulation according to claim 1, wherein the target insulator model is an insulator model with minimum electric field strength at a triple junction of metal, the insulation gas and the insulation material, and surface electric field strength less than a preset threshold.

5. The method for designing an insulator structure for mixed gas insulation according to claim 1, wherein the evaluating a thermodynamic parameter of the target insulator model based on conductivity-temperature variation curves of an insulation material and an insulation gas to obtain a temperature distribution of each part of the target insulator model specifically comprises:
calculating a temperature characteristic of the target insulator model based on the electric field distribution of each part of the target insulator model and the conductivity-temperature variation curves of the insulation material and the insulation gas to obtain the temperature distribution of each part of the target insulator model.

6. A device for designing an insulator structure for mixed gas insulation, comprising:
an insulator model generation module configured to obtain a plurality of sets of pre-set insulator structure parameters, and input each set of insulator structure parameters into an initial insulator model separately to generate a plurality of insulator models;
an electrical parameter evaluation module configured to evaluate an electrical parameter of each of the insulator models at a same temperature to obtain an electric field distribution of each part of each of the insulator models;
an insulator model screening module configured to select a target insulator model from all the insulator models based on a pre-defined model screening condition and the electric field distribution of each part of each of the insulator models;
a thermodynamic parameter evaluation module configured to evaluate a thermodynamic parameter of the target insulator model based on conductivity-temperature variation curves of an insulation material and an insulation gas to obtain a temperature distribution of each part of the target insulator model; and
an insulator model optimization module configured to optimize the target insulator model based on the temperature distribution of each part of the target insulator model to obtain an optimal insulator model, and obtain an insulator based on the optimal insulator model.

7. The device for designing an insulator structure for mixed gas insulation according to claim 6, wherein the electrical parameter evaluation module is specifically configured to: calculate tangential electric field strength, normal electric field strength, and total electric field strength of each of the insulator models separately at the same temperature to obtain the electric field distribution of each part of each of the insulator models.

8. The device for designing an insulator structure for mixed gas insulation according to claim 7, wherein the electrical parameter evaluation module comprises:
a tangential electric field strength calculation unit configured to: when it is detected that a surface charge accumulation of the insulator model reaches a maximum value, calculate a maximum value of a tangent vector of a surface electric field of the insulator model, and use the maximum value of the tangent vector of the surface electric field as the tangential electric field strength.

9. The device for designing an insulator structure for mixed gas insulation according to claim 6, wherein the target insulator model is an insulator model with minimum electric field strength at a triple junction of metal, the insulation gas and the insulation material, and surface electric field strength less than a preset threshold.

10. The device for designing an insulator structure for mixed gas insulation according to claim 6, wherein the thermodynamic parameter evaluation module is specifically configured to calculate a temperature characteristic of the target insulator model based on the electric field distribution of each part of the target insulator model and the conductivity-temperature variation curves of the insulation material and the insulation gas to obtain the temperature distribution of each part of the target insulator model.
